(19) United States
(12) Patent Application Publication
(10) Pub. No.: US 2017/0119962 A1
(43) Pub. Date: May 4, 2017

(54) SYRINGE IDENTIFICATION USING PHOTOCHROMIC INK

(71) Applicant: BAYER HEALTHCARE LLC, Whippany, NJ (US)

(72) Inventor: BRUNO FAZI, JR., PITTSBURGH, PA (US)

(21) Appl. No.: 15/319,006

(22) PCT Filed: Jun. 15, 2015
(86) PCT No.: PCT/US15/35780

§ 371 Date: Dec. 15, 2016

Related U.S. Application Data
(60) Provisional application No. 62/012,776, filed on Jun. 16, 2014.

(51) Int. Cl. A61M 5/145; A61M 5/00

(52) U.S. Cl. CPC A61M 5/14546; A61M 5/007; A61M 2205/6081; A61M 2205/6018; A61M 2205/6072

(57) ABSTRACT

A system for reading identifying information regarding a syringe assembly provided in a fluid injector is provided, the system including at least one syringe assembly having a syringe barrel with a distal end having a nozzle for expelling fluid therefrom and an open proximal end, the syringe barrel defining an interior volume, at least one indicator provided on at least a portion of an outer circumferential surface of the syringe barrel, the at least one indicator containing or associated with identifying information about the at least one syringe assembly, an injector having at least one syringe port configured to receive the at least one syringe assembly and to eject fluid therefrom, and a sensor module that activates the at least one indicator and receives information from the at least one indicator.

### CROSS REFERENCE TO RELATED APPLICATION

This application claims the benefit of U.S. Provisional Patent Application No. 62/012,776, filed Jun. 16, 2014, the disclosure of which is incorporated in its entirety by reference.

### BACKGROUND

Field of the Technology

The present disclosure relates generally to a system including at least one syringe for use with a fluid injector and, more specifically, a syringe including at least one indicator for identifying information about the at least one syringe.

Description of Related Art

In many medical procedures, such as drug delivery, it is desirable to inject various medical fluids into a patient. Likewise, numerous types of contrast media (often referred to simply as contrast) are injected into a patient for many diagnostic and therapeutic procedures, for example, imaging procedures, gene therapy, cell and biological agent delivery, and delivery of therapeutic agents generally. For example, contrast media are used in diagnostic imaging procedures such as X-ray procedures (including, for example, angiography, venography, and urography), computed tomography (CT) scanning, magnetic resonance imaging (MRI), and ultrasonic imaging. Contrast media are also used during therapeutic procedures, including, for example, angioplasty and other interventional radiological procedures.

In recent years, a number of injector-actuated syringes and powered injectors for pressurized injection of fluids, such as contrast media, have been developed. By way of example, in a typical angiographic procedure, the medical practitioner places a cardiac catheter into a vein or artery. The catheter is connected to either a manual or an automatic contrast injection mechanism. Alternatively, in CT and MRI imaging procedures, contrast may be injected into a patient's vasculature, for example through a needle or peripheral cannula. Such imaging agents may be accompanied by or followed by injection of a medical flushing fluid, such as saline. The automatic contrast injection mechanism typically includes at least one syringe connected to a powered injector having, for example, at least one powered linear actuator. Typically, an operator enters settings into an electronic control system of the powered injector for a fixed volume of contrast and a fixed rate of injection. In many systems, there is no interactive control between the operator and the powered injector, except to start or stop the injection. In these cases, the injector utilizes a programmed injection protocol to complete the procedure. A change in flow rate in such systems occurs by stopping the machine and resetting the injection parameters. Automation of angiographic procedures using powered injectors is discussed, for example, in U.S. Pat. Nos. 5,460,609, 5,573,515, and 5,800,397, incorporated herein by reference.

Generally, liquids being injected into a patient, such as X-ray and magnetic resonance contrast are clear pharmaceuticals delivered to a patient during a medical imaging procedure. Such contrasts can be detected by sophisticated imaging systems, but are difficult to detect with the human eye. Different contrasts have different properties, which make it difficult for a single or standard detector system to measure a property of the contrast. On the other hand, it is desirable that the contrast be clear to enable the user to check for particulates or other impurities before injecting the fluid into the patient. These impurities could be solid particles or improper chemicals with an identifiable color. For the visualization of particles, the contrast only needs to be transparent, but may be colored. For example, if X-ray contrast is stored improperly, free iodine can be released, causing the contrast to have a yellow tint.

It is often necessary for the medical practitioner to validate each syringe connected to the automatic fluid injection mechanism prior to performing the injection. Validation may include confirming that the syringe is genuine (e.g. to prevent counterfeiting) and determining various characteristics of the syringe and fluid contained therein. For example, the medical practitioner may verify that identifying information, such as the syringe dimensions (e.g. diameter, length, and fluid volume), and fluid contents are correct for the procedure being performed. In addition, the medical practitioner may be required to provide certain information about the syringe, such as frictional characteristics between the plunger and syringe barrel, fluid viscosity, and the like (referred to hereinafter as “injection parameters”) to the fluid injector to control piston force and acceleration to deliver fluid at a desired flow rate. Other important identifying information may include manufacturer, lot number, expiration date or shelf-life indicator, etc. Including the various desired identifying information may be difficult given the amount of available surface area and aesthetic reasons. For example, if the identifying information covers too much surface area on the syringe, it may impact the technician's ability to determine if the syringe has been properly filled.

In some systems, the fluid injector includes a sensor or reader located on the fluid injector for automatically reading a label or tag provided on the syringe when the syringe is inserted in the injector. The label or tag contains certain information about the syringe. The fluid injector uses the information extracted from the label or tag to validate the syringe and to control the injection. Some existing syringe identification procedures require specific labels or tags that are applied to the syringe subsequent to manufacturing the syringe. In certain cases, the medical practitioner must rotate the syringe to until the sensor or reader on the fluid injector is properly aligned with the syringe identification feature.

Therefore, there is a need for a syringe, injector, and/or injection system capable of identifying and validating the syringe prior to injection. The syringes, injectors, and systems set forth in the present disclosure are configured to address some or all of the above-identified problems.

### SUMMARY OF THE DISCLOSURE

The present disclosure generally relates to syringe and injector assemblies and to methods of identifying syringes used in the assemblies.

According to one aspect of the disclosure, a system for reading identifying information regarding a syringe assembly provided in a fluid injector is provided. The system may include at least one syringe assembly having a syringe barrel with a distal end having a nozzle for expelling fluid therefrom and an open proximal end, the syringe barrel defining an interior volume, at least one indicator provided on at least a portion of an outer circumferential surface of the syringe barrel, the at least one indicator containing or associated with identifying information about the at least one syringe assembly and having an inactive state and an active state upon application of electromagnetic radiation to the at least one indicator, an injector having at least one syringe port configured to receive the at least one syringe assembly and to eject fluid therefrom, and a sensor module that activates the at least one indicator and receives information from the at least one indicator.

The at least one indicator may include at least one photochromic ink. The identifying information about the at least one syringe assembly may include at least one of a syringe assembly type, physical dimensions of the at least one syringe assembly, flow characteristics of a fluid stored in the at least one syringe assembly, a type of fluid stored in the at least one syringe assembly, manufacturer of the at least one syringe assembly, lot number, date of manufacture of the at least one syringe assembly, expiration of use date of the at least one syringe assembly, and combinations thereof. The sensor module may include an electromagnetic radiation emitter unit that converts the at least one indicator from the inactive state to the active state and a detector unit that detects the identifying information about the at least one syringe assembly from the at least one indicator in the active state. The electromagnetic radiation emitter unit may emit ultra-violet light or other wavelength electromagnetic radiation to activate the at least one photochromic ink of the at least one indicator. The detector unit may detect a shade or color of the photochromic ink of the at least one indicator. The shade, color or wavelength of emitted electromagnetic radiation from the at least one photochromic ink may correspond to the identifying information about the at least one syringe assembly or alternatively, the pattern or shape of the at least one indicated may correspond to identifying information. A computer accessory or processor may be in communication with the sensor module where the computer may have software programming to interpret or “decode” the identifying information associated with the at least one indicator in the activated state. The at least one syringe assembly may include a plunger disposed within the interior volume. The plunger may be reversibly movable between the distal end and the proximal end of the at least one syringe assembly and include at least one second indicator positioned on at least a portion of an outer circumferential surface of the plunger. The sensor module may detect a location of the at least one indicator on the at least one syringe assembly and a location of the at least one second indicator on the plunger to determine a position of the plunger relative to the at least one syringe assembly. The at least one indicator may extend around the entire outer circumferential surface of the syringe barrel. The at least one indicator may be formed as at least one stripe, at least one geometric pattern, at least one symbol, letter, or number, or combinations of any thereof. The at least one indicator may be provided on the proximal end of the syringe barrel. The at least one indicator may be detected by the sensor module when the at least one syringe assembly is engaged within the at least one syringe port.

According to a further aspect of the disclosure, a syringe assembly is disclosed. The syringe assembly may include a syringe barrel extending from a distal end to a proximal end, the syringe barrel defining an interior volume, and at least one indicator including at least one photochromic ink provided on at least a portion of an outer circumferential surface of the syringe barrel.

The at least one indicator may contain or correspond to identifying information about the syringe assembly. The identifying information may include at least one of a syringe assembly type, physical dimensions of the syringe assembly, flow characteristics of a fluid stored in the syringe assembly, a type of fluid stored in the syringe assembly, manufacturer of the at least one syringe assembly, log number, date of manufacture of the at least one syringe assembly, expiration of use date of the at least one syringe assembly, and combinations thereof. A plunger may be disposed within the interior volume. The plunger may be reversibly movable between the distal end and the proximal end of the syringe barrel and include at least one second indicator provided on at least a portion of an outer circumferential surface of the plunger. The at least one indicator may extend around the entire outer circumferential surface of the syringe barrel. The at least one indicator may be formed as at least one stripe, at least one geometric patter, at least one symbol, letter, or number, or combinations of any thereof. The at least one indicator may be provided on the proximal end of the syringe barrel.

According to a further aspect of the disclosure, a method for reading identifying information regarding a syringe assembly provided within a fluid injector is disclosed. The method may include providing a syringe assembly having at least one indicator including at least one photochromic ink provided on a syringe barrel of the syringe assembly; inserting the syringe assembly into a syringe port of a fluid injector; activating the at least one photochromic ink of the at least one indicator on the syringe barrel using electromagnetic radiation from a sensor module of the fluid injector; and receiving identifying information regarding the syringe assembly from the at least one indicator using the sensor module. The method may further include providing a plunger within an interior volume defined by the syringe barrel, the plunger having at least one second indicator including at least one photochromic ink on at least a portion of an outer circumferential surface of the plunger; activating the at least one photochromic ink of the at least one second indicator on the plunger using electromagnetic radiation from the sensor module; and receiving information regarding a position of the plunger within the syringe barrel based on a location of the at least one second indicator on the plunger relative to a location of the at least one indicator on the syringe assembly.

Further details and advantages of various aspects described in detail herein will become clear upon reviewing the following detailed description of the various aspects in conjunction with the accompanying figures.

### BRIEF DESCRIPTION OF THE DRAWINGS

FIG. 1A is a perspective view of a fluid delivery system including a powered fluid injector and syringe according to an aspect of the disclosure;

FIG. 1B is a cross-sectional view of the powered injector and syringe of FIG. 1A;

FIG. 2 is a perspective view of the syringe for use with the powered injector of FIG. 1A according to an aspect of the disclosure;

FIG. 3 is a schematic cross-sectional view of a syringe port of the injector of FIG. 1A with the syringe of FIG. 1A inserted therein along with a sensor module according to an aspect of the disclosure; and

FIG. 4 is a cross-sectional view of a fluid delivery system including a syringe and a powered fluid injector according to an aspect of the disclosure.

### DESCRIPTION OF THE DISCLOSURE

The illustrations generally show preferred and non-limiting aspects of the systems and methods of the present disclosure. While the descriptions present various aspects of the devices, it should not be interpreted in any way as limiting the disclosure. Furthermore, modifications, concepts, and applications of the disclosure's aspects are to be interpreted by those skilled in the art as being encompassed, but not limited to, the illustrations and description provided herein.

The following description is provided to enable those skilled in the art to make and use the described aspects contemplated for carrying out the disclosure. Various modifications, equivalents, variations, and alternatives, however, will remain readily apparent to those skilled in the art. Any and all such modifications, variations, equivalents, and alternatives are intended to fall within the spirit and scope of the present disclosure.

Further, for purposes of the description hereinafter, the terms “end”, “upper”, “lower”, “right”, “left”, “vertical”, “horizontal”, “top”, “bottom”, “lateral”, “longitudinal”, and derivatives thereof shall relate to the disclosure as it is oriented in the figures. The term “proximal” refers to the direction towards the center or central region of the device. The term “distal” refers to the direction extending away from the central region of the device. However, it is to be understood that the disclosure may assume various alternative variations and step sequences, except where expressly specified to the contrary. It is also to be understood that the specific devices and processes illustrated in the attached drawings, and described in the following specification, are simply exemplary aspects of the disclosure. Hence, specific dimensions and other physical characteristics related to the aspects disclosed herein are not to be considered as limiting. For the purpose of facilitating understanding of the disclosure, the accompanying drawings and description illustrate preferred aspects thereof, from which the disclosure, various aspects of its structure, construction, and method of operation, and many advantages may be understood and appreciated.

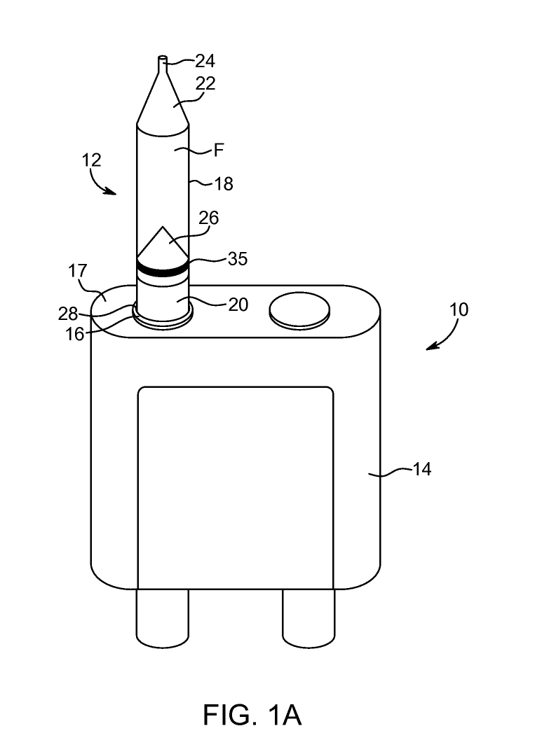

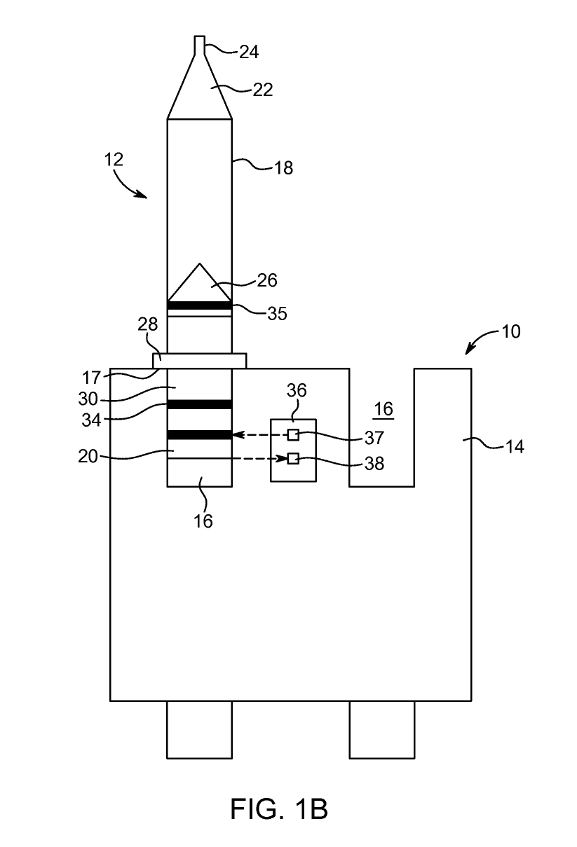

With reference to FIGS. 1A and 1B, an injector 10, such as an automated or powered fluid injector, is illustrated, which is adapted to interface with and actuate one or more syringes 12, which may be filled with a medical fluid F, such as contrast media, saline solution, or any desired medical fluid. The powered injector 10 may be used during a medical injection procedure to inject contrast and common flushing agents, such as saline, into the body of a patient prior to, during, and/or after an imaging procedure. The powered injector 10 is desirably at least a dual-syringe injector, wherein the two fluid delivery syringes 12 are oriented in a side-by-side or other relationship and are separately actuated by respective linear actuators or piston elements associated with the powered injector 10.

The injector 10 may be enclosed within a housing 14 formed from a suitable structural material, such as plastic or metal. The housing 14 may be in various shapes and sizes depending on the desired application. For example, the injector 10 may be a free-standing structure configured to be placed on the floor or may be a smaller design for placement on a suitable table or support frame. The injector 10 defines one or more syringe ports 16 for reversibly interfacing with syringes 12 and connecting the syringes 12 to respective piston elements (not shown). The syringe ports 16 are located on a top side of the housing 14. As will be described herein, the syringe 12 may include at least one indicator, such as at least one indicator stripe 34 (shown in FIG. 1B) at least one geometric pattern (such as a QR barcode, barcode, or other pattern), at least one symbol, letter, or number, or various combinations thereof, positioned on the syringe 12, which may be used to identify characteristics and other identifying information about the syringe 12, including one or more of the syringe type, physical dimensions, flow characteristics, fluid contents, manufacturer of the syringe, lot number, date of manufacture of the syringe, expiration of use date (i.e., the maximum shelf-life of the syringe), and similar information. The at least one indicator may be read by a corresponding sensor module 36 (shown in FIG. 1B), positioned on or recessed in the top side of the housing 14 of the injector 10.

A fluid path set (not shown) may be interfaced with the syringes 12 supported by the powered injector 10 for delivering fluid from the syringes 12 to a catheter (not shown) inserted into a patient at a vascular access site. For example, a flow of saline solution from one syringe and contrast from a second syringe may be regulated by a fluid control module (not shown). The fluid control module operates various valves and flow regulating structures to regulate the delivery of the saline solution and contrast to the patient based on user selected injection parameters, such as injection flow rate, duration, total injection volume, and ratio of contrast media and saline. A suitable multi-stage injector is described in U.S. patent application Ser. No. 13/386,765, filed on Jan. 24, 2012, and published as U.S. Patent Application Publication No. 2012/0123257, which is assigned to the assignee of the present application, the disclosure of which is incorporated herein by reference in its entirety. Other relevant multi-fluid delivery systems are found in U.S. patent application Ser. No. 10/159,592, filed on May 30, 2002 (published as U.S. Patent Application Publication No. 2004/0064041) and in U.S. patent application Ser. No. 10/722,370, filed Nov. 25, 2003 (published as U.S. Patent Application Publication No. 2005/0113754), both of which are assigned to the assignee of the present application, and the disclosures of which are incorporated herein by reference in their entirety. The principles of this disclosure may also be applied to fluid injectors that operate only a single syringe 12 or which operate more than two syringes 12.

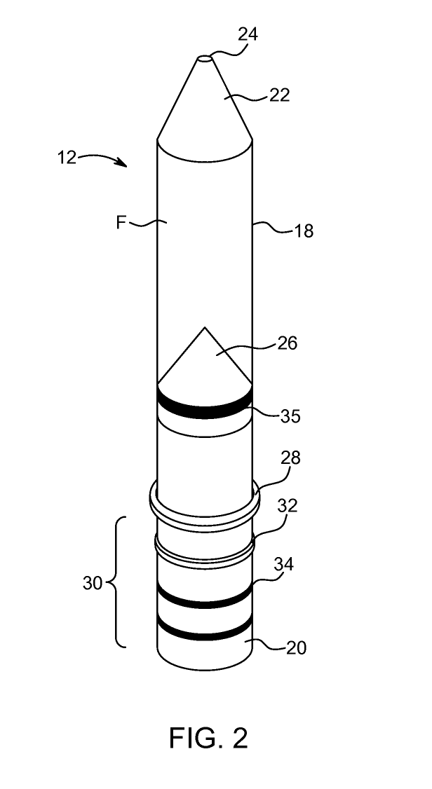

Having generally described the structure and function of the powered injector 10, the syringe 12 containing a fluid F for use with the injector 10, according to one aspect of the disclosure, is discussed in greater detail. With reference to FIG. 2, the syringe 12 generally may have a cylindrical syringe barrel 18 formed from glass or medical-grade plastic and defining an interior volume to store fluid F. The barrel 18 has an open proximal end 20 and a distal end 22 with a nozzle 24 extending distally therefrom. The open proximal end 20 may be sealed with a plunger, such as an elastomeric plunger 26 that is reversibly slidable through the barrel 18 between the proximal end 20 and the distal end 22 through the action of a piston activator associated with powered injector 10. The plunger 26 forms a liquid tight seal against the interior sidewall of the barrel 18 as the plunger 26 is advanced therethrough. An annular flange 28 may extend from the outer surface of the syringe barrel 18 at a position near the proximal end 20 of the barrel 18. When the syringe 12 is inserted into the injector 10 as shown in FIG. 1A, the flange, referred to hereinafter as a drip flange 28, rests against a distal opening 17 of the syringe port 16 to prevent excess fluid expelled from the syringe 12 from entering the syringe port 16 and fouling the interior workings of injector 10. The portion of the syringe barrel 18 between the drip flange 28 and the proximal end 20 of the syringe 12, referred to hereinafter as the insertion portion 30, may be sized and adapted to be inserted in the syringe port 16 of the injector 10 (as shown in FIG. 1B). Accordingly, in certain aspects, the insertion portion 30 of the barrel 18 includes one or more locking structures, such as a locking flange 32 (shown in FIG. 2), extending outward from the barrel 18. The locking flange 32 or other locking structure may be adapted to form a locking engagement with corresponding protrusions in the syringe port 16 of the injector 10 for reversibly holding the syringe 12 in the syringe port 16 while the injector 10 is in use. Alternatively, the insertion portion 30 may include various latches, locking mechanisms, or radially extending ribs for connection to corresponding portions of the syringe port 16. A suitable syringe for use with the injector 10 depicted in FIGS. 1A and 1B may be found in U.S. Pat. No. 5,383,858, issued Jan. 24, 1995, and which is assigned to the assignee of the present application, the disclosure of which is incorporated herein in its entirety. Other relevant syringes may be found in U.S. Pat. No. 6,322,535, issued Nov. 27, 2001, and in U.S. Pat. No. 6,652,489, issued Nov. 25, 2003, both of which are assigned to the assignee of the present application, and the disclosures of which are both incorporated herein by reference in their entirety. Other suitable locking mechanisms are described in U.S. patent application Ser. No. 14/526,294, filed Oct. 28, 2014, and Ser. No. 14/526,395, filed Oct. 28, 2014.

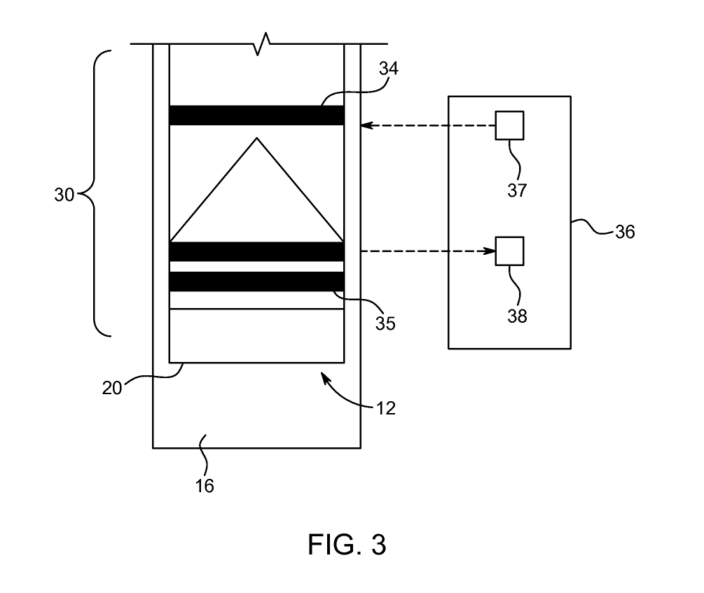

With reference to FIG. 3, the syringe 12, in one aspect, includes at least one first indicator stripe 34 positioned on the insertion portion 30 of the syringe 12. It is also contemplated that additional indicator stripes may be provided on the syringe 12. According to other embodiments, the at least one indicator may have a shape other than a stripe, for example at least one geometric pattern, at least one symbol, at least one letter, at least one number, or various combinations of these indicator shapes. As used herein, the term “indicator stripe” also may include other indicator shapes. Examples of indicator shapes or designs include QR bar codes, other bar code designs, UPC codes, manufacturer names and/or trademarks or symbols, and various number/letter patterns, for example patterns corresponding to encoded identifying information. The first indicator stripe 34 may extend continuously or discontinuously around at least a portion of the outer circumferential surface of the syringe 12. In another aspect, the first indicator stripe 34 may extend around the entire outer circumferential surface of the syringe 12. In another aspect, at least one second indicator stripe 35 is positioned on the plunger 26 of the syringe 12. The second indicator stripe 35 may be provided around at least a portion of an outer circumferential surface of the plunger 26. In other aspects, one or more indicator stripe 34 may be on the outer circumferential surface of the syringe 12 and one or more indicator stripe 35 may be on an outer surface of the plunger 26. In one aspect, at least one of the first and second indicator stripes 34, 35 may have at least one photochromic ink. In one aspect, photochromic ink may be ink that changes color, shades of color, wavelength of emitted electromagnetic radiation, shape or pattern when exposed to ultra-violet light or electromagnetic radiation having a specific wavelength equal to an absorption wavelength of the at least one photochromic ink. The at least one photochromic ink may have a first inactive state and a second active state, wherein the at least one photochromic ink transitions from the first inactive state to the second active state upon absorption of the electromagnetic radiation of a specified wavelength. In certain embodiments, the photochromic ink may transition back from the second active state to the first inactive state when the electromagnetic radiation of the specific wavelength is removed. The transition from the second active state to the first inactive state may proceed by a phosphorescence or fluorescence mechanism accompanied by the emission of electromagnetic radiation from the photochromic ink having a different, longer wavelength than the specified wavelength of the absorbed electromagnetic radiation. In other embodiments, the at least one photochromic ink may remain in the active state or transition to a second permanent color after removal of the electromagnetic radiation. The indicator stripe 34 with photochromic ink may be integrally molded on the syringe 12 during the molding process of the syringe 12 or, alternatively, may be applied or painted on to the outer surface of the syringe 12 after the syringe 12 has been formed. In some aspects, the syringe 12 may only include the first indicator stripe 34 on the insertion portion 30, only the second indicator stripe 35 on the plunger 26, or both the first indicator stripe 34 on the insertion portion 30 and the second indicator stripe 35 on the plunger 26. The first and/or second indicator stripes 34, 35 may be configured to be detected and read by a sensor module positioned in the injector 10, such as the sensor module 36 depicted in FIG. 1B. The sensor module 36 may include an electromagnetic radiation emitter unit 37 and a detector unit 38, the functions of which are described herein. The electromagnetic radiation emitter unit 37 may be configured to emit electromagnetic radiation of a specified wavelength, for example ultra-violet electromagnetic radiation, from the sensor module 36, which activates the at least one photochromic ink of at least one of the first and/or second indicator stripes 34, 35 causing the at least one photochromic ink to illuminate to a certain degree as described herein. The electromagnetic radiation emitter unit 37 may be a light bulb or any other source of electromagnetic energy, such as, for example a laser. The detector unit 38 may be configured to detect a specific shade, color, wavelength of emitted electromagnetic radiation, shape or pattern of the at least one photochromic ink of the indicator in the second active state, such as from the first and second indicator stripes 34, 35 as the syringe 12 is inserted into the injector 10 and irradiated by the electromagnetic radiation emitter unit 37. The detector unit 38 may be a camera, an electromagnetic radiation sensor, such as an ultra-violet sensor, or any other device capable of detecting a change in color, shade, wavelength of emitted electromagnetic radiation, shape or pattern of the at least one photochromic ink on the indicator. In specific embodiments, the sensor module 36 may comprise a separate electromagnetic radiation emitter unit 37 and a separate detector unit 38, for example, located at different locations within the injector assembly 14.

The first and optional second indicator stripes 34, 35 may be associated with identifying information about the syringe assembly, the syringe 12 and/or the fluid F contained or to be contained therein. For example, the information may correspond to the type of syringe 12, the physical dimensions of the syringe 12 (e.g., length and diameter, fluid volume, nozzle dimensions, etc.) or other information, such as, but not limited to, flow characteristics of a fluid stored in the at least one syringe assembly, a type of fluid stored in the at least one syringe assembly, manufacturer of the at least one syringe assembly, lot number, date of manufacture of the at least one syringe assembly, expiration of use date of the at least one syringe assembly, and combinations thereof. In addition, the first and/or second indicator stripes 34, 35 may include identifying information corresponding to physical parameter information for the syringe 12, including barrel/plunger friction characteristics, pressure limitations, maximum or minimum flow rates, and fluid type. The physical parameter information and other identifying information about the syringe 12 may be used to determine a preferred injection force, injection velocity, and appropriate power level for the linear actuator of the injector 10. Examples of types of relevant information that may be communicated or transmitted between a syringe and injector is discussed in U.S. Pat. No. 6,743,202, issued on Jun. 1, 2004, and which is assigned to the assignee of the present application, the disclosure of which is incorporated herein by reference in its entirety. The information corresponding to the syringe 12 is identified according to the specific shade, color, wavelength of emitted electromagnetic radiation, shape or pattern emitted by the at least one photochromic ink of the first and/or second indicator stripes 34, 35 in the activated state, which is detected and interpreted by the detector unit 38. For example, a green color for the photochromic ink may correspond to the physical dimensions of the syringe 12, while a red color for the photochromic ink may correspond to the fluid contents of the syringe 12. Alternatively, a darker shade of color for the photochromic ink may correspond to the physical dimensions of the syringe 12, while a lighter shade of color for the photochromic ink may correspond to the fluid contents of the syringe 12.

The first and/or second indicator stripes 34, 35 may be provided in various shapes, depending on the shape and size of the insertion portion 30, the injector 10, and/or syringe port 16. For example, the first and/or second indicator stripes 34, 35 may be a single square or rectangular label positioned on the insertion portion 30 of the syringe 12. Alternatively, the first and/or second indicator stripes 34, 35 may include dotted lines of photochromic ink. Further, the first and/or second indicator stripes 34, 35 may be polygonal, such as triangular-shaped, or circular-shaped. It is also contemplated that more than one first and second indicator stripe 34, 35 may be positioned on the insertion portion 30 and/or plunger 26, respectively. In various embodiments, the first indicator stripe 34 may have a different size, shape, and/or design than the second indicator stripe 35. In this aspect, additional information or redundant information regarding the unique features of the syringe 12 may be conveyed to the operator.

In addition to providing identifying information about the syringe 12 and/or plunger 26, the first and/or second indicator stripes 34, 35 may also be configured to convey information about the position of the syringe 12 relative to the syringe port 16 or the position of the plunger 26 relative to the syringe 12. For example, the first and/or second indicator stripes 34, 35 may convey that the syringe 12 has been correctly inserted into the syringe port 16. Specifically, the injector 10 may alert the user anytime that a portion of the first and/or second indicator stripes 34, 35 is identified by the sensor module 36 in the injector 10 and indicate whether the syringe is a compatible syringe or that the syringe has been correctly inserted. In other aspects, the first and/or second indicator stripes 34, 35 may identify when the syringe 12 is in the “home” or ready-for-use position within the syringe port 16, for example, indicating that the plunger 26 is in the fully proximal position in syringe 12 and ready to interface with the piston assembly of injector 10 for the start of the fluid injection procedure. Thus, the first and/or second indicator stripes 34, 35 may supplement or replace various physical positioning structures extending from the syringe barrel 18 for positioning the syringe 12 within the syringe port 16. Accordingly, in various aspects the syringe 12 may be less complex and easier to manufacture than conventional syringes, which may include such positioning structures. It is also contemplated that the position of the plunger 26 relative to the syringe 12 may be conveyed to the sensor module 36 based on the color emitted from the combined at least one photochromic ink of the first and second indicator stripes 34, 35 as they overlap one another. For example, as the plunger 26 moves towards either the proximal end 20 or the distal end 22 of the syringe 12, the second indicator stripe 35 of the plunger may pass over at least one first indicator stripe 34 positioned on the insertion portion 30 of the syringe 12, thereby conveying the position of the plunger 26 in the syringe 12.

Having described the structure of the syringe 12 and first and/or second indicator stripes 34, 35, the interaction between the syringe 12, the first and second indicator stripes 34, 35, and the sensor module 36 is described in detail. As shown in FIG. 3, the injector 10 includes a sensor module 36 including the electromagnetic radiation emitter unit 37 and the detector unit 38 configured to locate, identify, and interpret the specific color, shade, wavelength of emitted electromagnetic radiation, shape or pattern of the first and/or second indicator stripes 34, 35 for example in the activated state. The sensor module 36 may be positioned adjacent to the syringe port 16 of the injector 10. In certain aspects, the sensor may be recessed below the surface of the syringe port 16 of the injector 10. As the insertion portion 30 of the syringe 12 is inserted into the syringe port 16, the electromagnetic radiation emitter unit 37 emits electromagnetic radiation, such as ultra-violet light, into the syringe port 16. As the insertion portion 30 moves further into the syringe port 16, the first and/or second indicator stripes 34, 35 of the syringe 12 and/or the plunger 26 will pass through the electromagnetic radiation from the emitter 37. As this occurs, the at least one photochromic ink of the first and/or second indicator stripes 34, 35 will illuminate or change to a specific color, shade, wavelength of emitted electromagnetic radiation, shape or pattern as it transitions to the second active state. This specific color, shade, wavelength of emitted electromagnetic radiation, shape or pattern corresponds to specific identifying information regarding the syringe 12 and/or the plunger 26. As the first and/or second indicator stripes 34, 35 change to a specific color, shade, wavelength of emitted electromagnetic radiation, shape or pattern, the detector unit 38 detects which specific color, shade, wavelength of emitted electromagnetic radiation, shape or pattern is emitted from the first and/or second indicator stripes 34, 35 and relays this corresponding information to the sensor module 36.

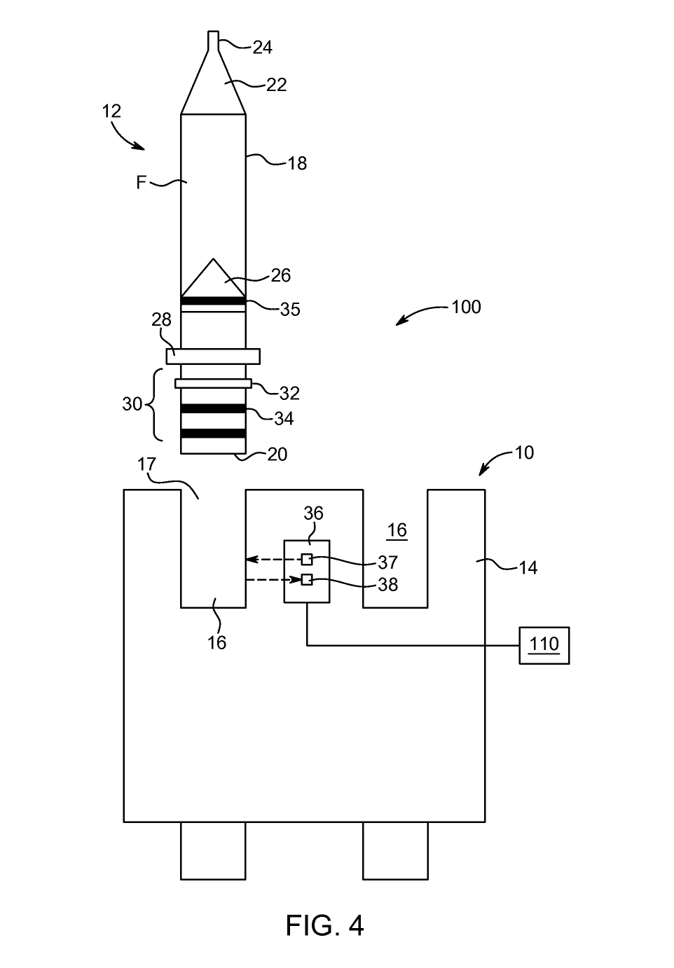

With reference to FIG. 4, a fluid delivery system 100 including the at least one syringe 12, at least one first and/or second indicator stripes 34, 35, fluid injector 10 having at least one syringe port 16, and sensor module 36, is illustrated. In use, an operator may insert the proximal end 20 of the syringe 12 into the syringe port 16. The operator may be required to exert some force against the syringe 12 so that the locking flange 32 of the syringe 12 engages with the corresponding locking structures (not shown) of the syringe port 16 to form a suitable, reversible connection therewith. The operator continues to press the syringe 12 into the syringe port 16 until the insertion portion 30 of the syringe barrel 18 is properly seated in operative connection with the fluid injector 10. Once the syringe 12 is inserted in the syringe port 16, in certain embodiments the operator may twist or rotate the syringe 12 relative to the syringe port 16 until the syringe 12 is in the home or ready-for-use position. In other embodiments, the syringe may self-orient itself in the proper position. In still other embodiments, the injector 10 may include a plurality of sensor modules 36 which can detect the at least one indicator regardless of the orientation. It is also contemplated that the operator may just push the syringe 12 relative to the syringe port 16 to insert the syringe 12 in the home or ready-for-use position. In this aspect, the indicator stripes 34, 35 are positioned around the entire circumferential surface of the syringe 12 and/or plunger 26, respectively, so that the indicator strips 34, 35 may be detected by the sensor module 36 at any angle relative to the syringe 12 and/or plunger 26. Inserting the insertion portion 30 of the syringe 12 into the syringe port 16 brings the indicator stripes 34, 35 into contact with the electromagnetic radiation that is being emitted from the electromagnetic radiation emitter unit 37 of the sensor module 36. The corresponding information conveyed by the specific colors, shades, wavelength of emitted electromagnetic radiation, shape or pattern of the at least one first and/or second indicator stripes 34, 35 is sent to or detected by the detector unit 38 and, thereby, sent to the sensor module 36. The sensor module 36 may then relay this information to one or more computer accessory or processor 110, such as a computer processor, a computer terminal, injector software, hospital information network, or a portable electronic device, by a wired or wireless connection, associated with the injector 10, for example to provide a visual or audible alert regarding the identifying information about the syringe 12. This information may be used by the injector 10 to modify or adapt the injector settings prior to initiating the injection process or notify a user of a potential problem (e.g., incorrect syringe, expired syringe etc.). Similarly, the injector 10 may be configured to cancel the injection process if the loaded syringe 12 and/or fluid contained therein is inappropriate for the procedure to be performed. Assuming that the syringe and fluid are correct, the injector 10 may automatically begin an injection procedure, optionally using a portion of the information contained in the at least one indicator. Alternatively, the operator may be required to initiate the injection procedure by an actuation activity, such as pressing a start button. Once the injection procedure begins, the linear actuator of the injector 10 contacts and engages the proximal end of the plunger 26 disposed within the syringe barrel 18, for example to initiate filling of an empty syringe or begin injection for a prefilled syringe. Once the at least one syringe 12 is filled with fluid, movement of the plunger 26 in the distal direction of the syringe barrel 18 expels fluid contained within the syringe 12, thereby injecting fluid through a tubing assembly into the patient through any known injection structure, such as an IV tube or needle accessory.

Although the discussion has been described in detail for the purpose of illustration based on what is currently considered to be the most practical and preferred aspects, it is to be understood that such detail is solely for that purpose and that the disclosure is not limited to the disclosed aspects, but, on the contrary, is intended to cover modifications and equivalent arrangements. For example, it is to be understood that the present disclosure contemplates that, to the extent possible, one or more features of any aspect can be combined with one or more features of any other aspect.

1. A system for reading identifying information regarding a syringe assembly provided in a fluid injector, the system comprising:
at least one syringe assembly having a syringe barrel with a distal end having a nozzle for expelling fluid therefrom and an open proximal end, the syringe barrel defining an interior volume;
at least one indicator provided on at least a portion of an outer circumferential surface of the syringe barrel, the at least one indicator containing or corresponding to identifying information about the at least one syringe assembly and having an inactive state and an active state upon application of electromagnetic radiation to the at least one indicator,
an injector having at least one syringe port configured to receive the at least one syringe assembly and to eject fluid therefrom; and
a sensor module that activates the at least one indicator and receives information from the at least one indicator.

2. The system of claim 1, wherein the at least one indicator comprises at least one photochromic ink.

3. The system of claim 1, wherein the identifying information about the at least one syringe assembly includes at least one of a syringe assembly type, physical dimensions of the at least one syringe assembly, flow characteristics of a fluid stored in the at least one syringe assembly, a type of fluid stored in the at least one syringe assembly, manufacturer of the at least one syringe assembly, lot number of the at least one syringe assembly, date of manufacture of the at least one syringe assembly, expiration of use date of the at least one syringe assembly, and combinations thereof.

4. The system of claim 1, wherein the sensor module comprises an electromagnetic radiation emitter unit that converts the at least one indicator from the inactive state to the active state and a detector unit that detects the identifying information about the at least one syringe assembly from the at least one indicator in the active state.

5. The system of claim 4, wherein the at least one indicator comprises at least one photochromic ink,
wherein the electromagnetic radiation emitter unit emits ultra-violet light to activate the at least one photochromic ink of the at least one indicator, and
wherein the detector unit detects a shade, color, wavelength of emitted electromagnetic radiation, shape or pattern of the activated at least one photochromic ink of the at least one indicator, wherein the shade, color, wavelength of emitted electromagnetic radiation, shape or pattern of the at least one photochromic ink corresponds to the identifying information about the at least one syringe assembly.

6. The system of claim 1, further comprising a computer accessory in communication with the sensor module.

7. The system of claim 1, wherein the at least one syringe assembly comprises a plunger disposed within the interior volume,
wherein the plunger is reversibly movable between the distal end and the proximal end of the at least one syringe assembly and includes at least one second indicator positioned on at least a portion of an outer circumferential surface of the plunger.

8. The system of claim 7, wherein the sensor module detects a location of the at least one indicator on the at least one syringe assembly and a location of the at least one second indicator on the plunger to determine a position of the plunger relative to the at least one syringe assembly.

9. The system of claim 1, wherein the at least one indicator extends around the entire outer circumferential surface of the syringe barrel.

10. The system of claim 1, wherein the at least one indicator is formed as at least one stripe, at least one geometric pattern, at least one symbol, letter or number, or combinations of any thereof.

11. The system of claim 1, wherein the at least one indicator is provided near the proximal end of the syringe barrel.

12. The system of claim 1, wherein the at least one indicator is detectable by the sensor module when the at least one syringe assembly is engaged within the at least one syringe port.

13. A syringe assembly, comprising:
a syringe barrel extending from a distal end to a proximal end, the syringe barrel defining an interior volume, and
at least one indicator comprising at least one photochromic ink provided on at least a portion of an outer circumferential surface of the syringe barrel.

14. The syringe assembly of claim 13, wherein the at least one indicator contains or corresponds to identifying information about the syringe assembly, and
wherein the identifying information includes at least one of a syringe assembly type, physical dimensions of the syringe assembly, flow characteristics of a fluid stored in the syringe assembly, a type of fluid stored in the syringe assembly, manufacturer of the at least one syringe assembly, lot number of the at least one syringe assembly, date of manufacture of the at least one syringe assembly, expiration of use date of the at least one syringe assembly, and combinations thereof.

15. The syringe assembly of claim 13, further comprising a plunger disposed within the interior volume,
wherein the plunger is reversibly movable between the distal end and the proximal end of the syringe barrel and includes at least one second indicator provided on at least a portion of an outer circumferential surface of the plunger.

16. The syringe assembly of claim 13, wherein the at least one indicator extends around the entire outer circumferential surface of the syringe barrel.

17. The syringe assembly of claim 13, wherein the at least one indicator is formed as at least one stripe, at least one geometric pattern, at least one symbol, letter or number, or combinations of any thereof.

18. The syringe assembly of claim 13, wherein the at least one indicator is provided near the proximal end of the syringe barrel.

19. A method for reading identifying information regarding a syringe assembly engaged with a fluid injector, the method comprising the steps of:
providing a syringe assembly having at least one indicator comprising at least one photochromic ink provided on a syringe barrel of the syringe assembly;
inserting the syringe assembly into a syringe port of the fluid injector;
activating the at least one photochromic ink of the at least one indicator on the syringe barrel using electromagnetic radiation from a sensor module of the fluid injector; and
receiving identifying information regarding the syringe assembly from the at least one indicator using the sensor module.

20. The method of claim 19, further comprising:
providing a plunger within an interior volume defined by the syringe barrel, the plunger having at least one second indicator comprising at least one second photochromic ink on at least a portion of an outer circumferential surface of the plunger,
activating the at least one second photochromic ink of the at least one second indicator on the plunger using electromagnetic radiation from the sensor module, and
receiving information regarding a position of the plunger within the syringe barrel based on a location of the at least one second indicator on the plunger relative to a location of the at least one indicator on the syringe assembly.
